(19) United States
(12) Patent Application Publication
(10) Pub. No.: US 2017/0253420 A1
(43) Pub. Date: Sep. 7, 2017

(54) BLOOD TEST STRIP DISPENSER

(71) Applicant: Michael E. Smith, Columbia, TN (US)

(72) Inventor: Michael E. Smith, Columbia, TN (US)

(21) Appl. No.: 15/448,382

(22) Filed: Mar. 2, 2017

Related U.S. Application Data
(60) Provisional application No. 62/302,528, filed on Mar. 2, 2016.

(51) Int. Cl. B65D 83/08; G01N 33/49; B65D 85/62; G01N 33/487

(52) U.S. Cl. CPC B65D 83/0829; G01N 33/48757; G01N 33/49; B65D 85/62

(57) ABSTRACT

A blood test strip dispenser is provided. The dispenser includes a body having an inner volume and an aperture extending from the inner volume through an outer surface of the body. A plurality of blood test strips are temporarily retained within the inner volume of the body of the dispenser. An actuating device operates to dispense a single blood test strip from within the inner volume through the aperture. A lifting device operates to lift the remaining blood test strips of the plurality of blood test strips after the single blood test strip has been dispensed. Lifting the remaining blood test strips aligns the top blood test strip of the remaining blood test strips with the aperture.

### CROSS REFERENCE TO RELATED APPLICATION[S]

This application claims priority to U.S. Provisional Patent Application entitled “BLOOD TEST STRIP DISPENSER,” Ser. No. 62/302,528, filed Mar. 2, 2016, the disclosure of which is hereby incorporated entirely herein by reference.

### BACKGROUND OF THE INVENTION

Technical Field

This invention relates generally to containers for blood test strips and more particularly to a dispenser for blood test strips.

State of the Art

Millions of people have been diagnosed with diabetes. For these individuals, they must live with this condition and learn to test and treat themselves. One of the tasks for one having diabetes is to regularly check the glucose levels in the blood. Glucose testing is an important and vital part of a diabetic person's daily health care. Glucose testing is done by using a glucose testing meter, which uses a blood test strip or also referred to as a glucose test strip. To test for glucose a strip is placed within a glucose meter and the individual must drop a sample of blood on the strip. This can be done by pricking the skin on a finger to allow the sample of blood to flow right onto the glucose strip. Once the blood sample has made it on to the glucose strip, the glucose meter is used to measure the glucose in the blood. In each test strip, there is a chemical called glucose oxidase that reacts with the glucose in the blood sample and creates gluconic acid. The glucose meter operates to read and determine how much glucose is in the sample of blood on the testing strip. The number is then relayed on the screen of the glucose testing meter.

The blood test strips must be handled with care and stored with care in order to ensure their proper function. Currently, containers for test strips may hold several test strips and a user would need to ensure the hands are clean, remove the lid to the container and separate the necessary strip, much like sorting through pill medication in a bottle. Other devices may utilize a drum; however, the drum is only useable within particular glucose meters.

There is not a dispenser capable of dispensing a single test strip at a time to be used with any glucose meter. Accordingly, there is a need for an improved blood test strip dispenser.

### SUMMARY OF THE INVENTION

The present invention relates to a blood test strip dispenser for storing a plurality of test strips and including a dispensing mechanism for dispensing a single test strip from the plurality of test strips.

An embodiment of the present invention includes a blood test strip dispenser comprising a body having an inner volume and an aperture extending from the inner volume through an outer surface of the body; a plurality of blood test strips removably retained within the inner volume of the body; an actuating device for dispensing a single blood test strip of the plurality of blood test strips within the inner volume through the aperture, wherein the single blood test strip is a top blood test strip of the plurality of blood test strips; and a lifting device for lifting the remaining blood test strips of the plurality of blood test strips after the single blood test strip has been dispensed, wherein lifting the remaining blood test strips comprises aligning a top blood test strip of the remaining blood test strips with the aperture.

Another embodiment may include a blood test kit comprising: a blood test strip dispenser; and a plurality of blood test strips, wherein the plurality of blood test strips are pre-loaded within the blood test strip dispenser or are provided in the kit for loading by a user, and wherein the blood test strip dispenser comprises: a body having an inner volume and an aperture extending from the inner volume through an outer surface of the body, the plurality of blood test strips removably retained within the inner volume of the body; an actuating device for dispensing a single blood test strip of the plurality of blood test strips within the inner volume through the aperture, wherein the single blood test strip is a top blood test strip of the plurality of blood test strips; and a lifting device for lifting the remaining blood test strips of the plurality of blood test strips after the single blood test strip has been dispensed, wherein lifting the remaining blood test strips comprises aligning a top blood test strip of the remaining blood test strips with the aperture.

Yet another embodiment includes a method of using a blood test strip dispenser, the method comprising: automatically aligning a top blood test strip of a plurality of blood test strips within the blood test strip dispenser with a dispensing aperture of the blood test strip dispenser; operating an actuating device to extend the top blood test strip through the dispensing aperture; removing the top blood test strip from the blood test strip dispenser; automatically lifting the remaining blood test strips toward a top side of the blood test strip dispenser; and automatically aligning a top blood test strip of the remaining blood test strips with the dispensing aperture.

The foregoing and other features and advantages of the present invention will be apparent from the following more detailed description of the particular embodiments of the invention, as illustrated in the accompanying drawings.

### BRIEF DESCRIPTION OF THE DRAWINGS

A more complete understanding of the present invention may be derived by referring to the detailed description and claims when considered in connection with the Figures, wherein like reference numbers refer to similar items throughout the Figures, and:

FIG. 1 is a perspective view of a blood test strip dispenser in accordance with embodiments;

FIG. 2 is a side section view of a blood test strip dispenser with the dispensing mechanism in a stored condition in accordance with embodiments;

FIG. 3 is a side section view of a blood test strip dispenser with the dispensing mechanism in a dispensing condition in accordance with embodiments; and

FIG. 4 is a flow chart of a method of using a blood test strip dispenser, in accordance with embodiments.

### DETAILED DESCRIPTION OF EMBODIMENTS OF THE INVENTION

As discussed above, embodiments of the present invention relate to a blood test strip dispenser for storing a plurality of test strips and including a dispensing mechanism for dispensing a single test strip from the plurality of test strips.

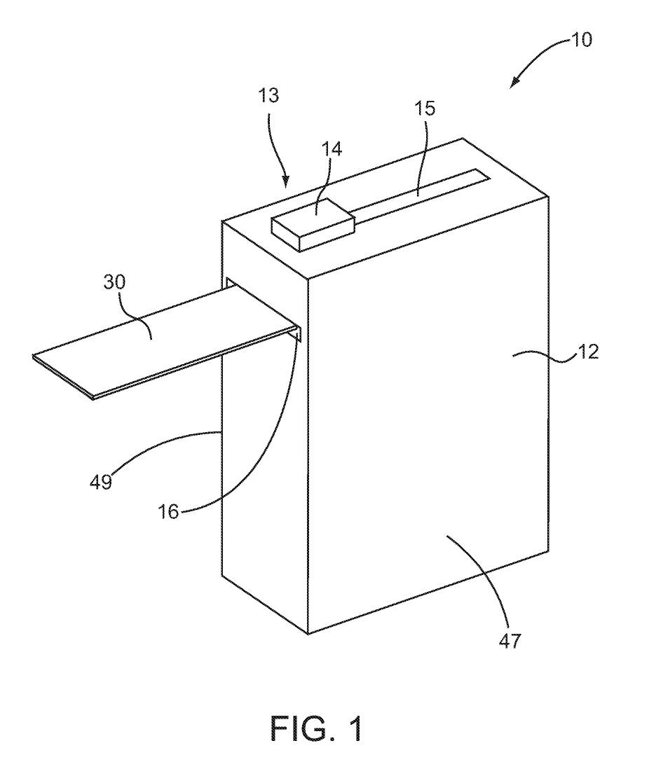

Referring to FIGS. 1-3, a blood test strip dispenser 10 is depicted in accordance with embodiments of the present invention. The dispenser 10 may include a body 12 having an inner volume 26. The body 12 may have a first side 40, a second side 42, a bottom side 46, a top side 48 and two side faces 47 and 49. The inner volume 26 may be a size and shape to hold and retain a plurality of test strips 30. The inner volume may be bound by the first side 40, the second side 42, the bottom side 46, the top side 48 and the two side faces 47 and 49. The dispenser 10 further includes a dispensing mechanism formed of an actuating device 13 and a lifting device 20.

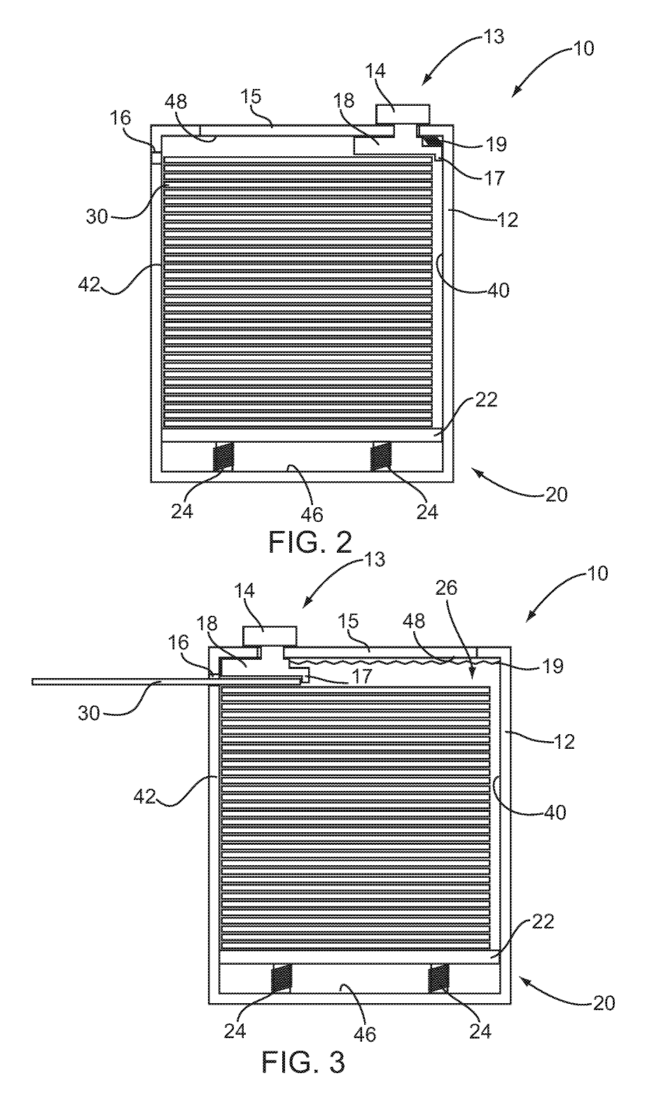

The actuating device 13 may include a control slider 14, a strip engagement member 18, a protrusion 17 and a biasing member 19, wherein the control slider 14 is coupled to the strip engagement member 18 through a slot 15 formed in the top side 48 of the body 12. As shown in FIG. 2, the actuating device 13 is biased toward a first side 40 by biasing member 19, placing the dispenser 10 in a stored position, wherein the plurality of strips 30 are within the inner volume 26 of the dispenser 10. Applying a force on the control slider 14 in a direction depicted in FIG. 3 as arrow 44 moves the strip engagement member 18 having protrusion 17 toward the second side 42 of the body 12 of the dispenser 10. Protrusion 17 engages an end of the top test strip 30 of the plurality of test strips 30 and extends the top test strip 30 through an aperture 16 formed in the second side 42 of the body 12 of the dispenser 10, thereby placing the dispenser 10 in a dispensing condition. The test strip 30 may then be inserted within a glucose meter directly from the dispenser 10, thereby reducing possible contamination of the test strip 30. The protrusion 17 has a length to only engage an end of one test strip 30 at a time.

Once the test strip 30 is inserted within the glucose meter, the force applied to the control slider 14 may be released and the biasing member 19 returns the actuating device 13 toward the first side 40, thereby placing the dispenser 10 in the stored condition. When in the stored condition, and after dispensing the top strip 30, the lifting device 20 may then operate. The lifting device 20 may include a support member 22 and at least one lift biasing member 24. The support member 22 supports the plurality of test strips 30 within the inner volume 26 of the body 12 of the dispenser 10. The at least one biasing member 24 may operate to bias the support member 22 away from the bottom side 46 of the body 12 and toward the top side 48 of the body of the dispenser 10. Accordingly, when a strip 30 is dispensed from the dispenser 10, the lifting device 20 lifts the plurality of strips 30 toward the top side 48 and aligns the top strip 30 with the aperture 16 of the body 12.

In embodiments, the dispenser 10 may be provided as part of a blood test kit from a manufacturer with blood test strips pre-loaded with in the dispenser 10 in order to reduce the possibility of contamination. In these embodiments, the dispenser 10 with the pre-loaded test strips may be disposable after all of the strips are used. In other embodiments, the dispenser 10 may be provided as part of a blood test kit with blood test strips also provided as part of the kit, wherein the inner volume 26 of the body 12 of the dispenser 10 may be accessible by a user of the test strips and the user may load the dispenser 10 with the test strips.

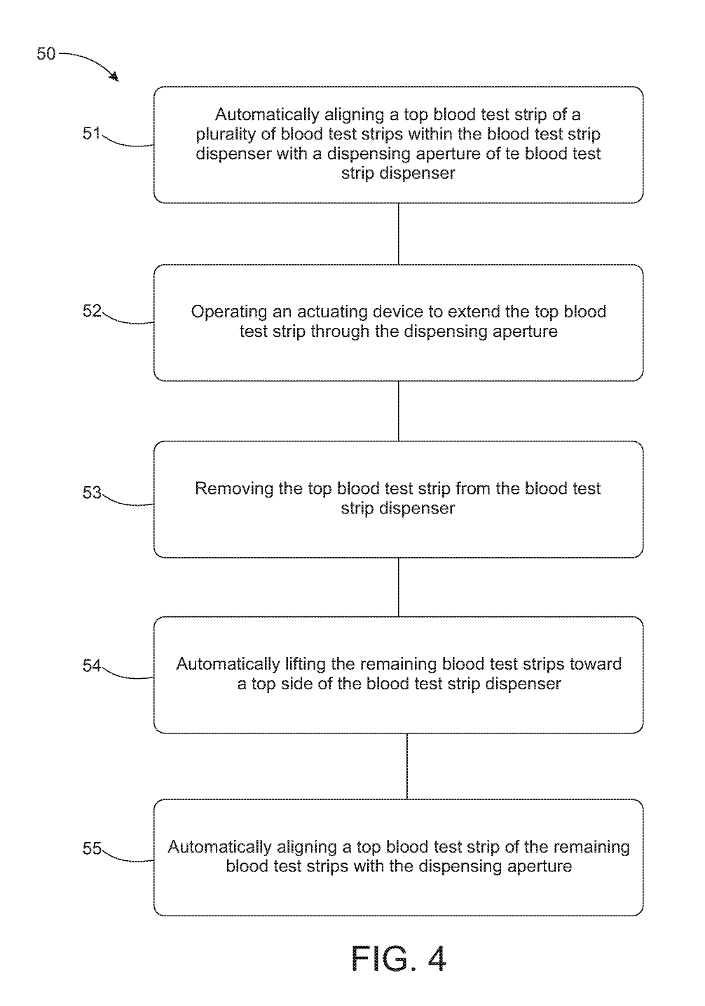

Referring to FIG. 4, another embodiment includes a method 50 of using a blood test strip dispenser. The method 50 includes automatically aligning a top blood test strip of a plurality of blood test strips within the blood test strip dispenser with a dispensing aperture of the blood test strip dispenser (Step 51); operating an actuating device to extend the top blood test strip through the dispensing aperture (Step 52); removing the top blood test strip from the blood test strip dispenser (Step 53); automatically lifting the remaining blood test strips toward a top side of the blood test strip dispenser (Step 54); and automatically aligning a top blood test strip of the remaining blood test strips with the dispensing aperture (Step 55).

In method 50, Step 52 of operating the actuating device may include automatically engaging the top blood test strip of the plurality of blood test stripes within the blood test strip dispenser. Further, Step 53 of removing the top blood test strip may include inserting the extended blood test strip within a glucose meter directly from the blood test strip dispenser to reduce possible contamination of the extended blood test strip.

It will be understood that embodiments of a test strip dispenser are not limited to the specific components disclosed herein, and are not limited to uses of any specific components, provided that the components selected are consistent with the intended operation of a method and/or system for dispensing blood test strips.

Accordingly, the components defining any blood test strip dispenser may be formed of any of many different types of materials or combinations thereof that can readily be formed into shaped objects provided that the components selected are consistent with the intended operation of a blood test strip dispenser. For example, the components may be formed of: rubbers (synthetic and/or natural) and/or other like materials; glasses (such as fiberglass) carbon-fiber, aramid-fiber, any combination thereof, and/or other like materials; polymers such as thermoplastics (such as ABS, Fluoropolymers, Polyacetal, Polyamide; Polycarbonate, Polyethylene, Polysulfone, and/or the like), thermosets (such as Epoxy, Phenolic Resin, Polyimide, Polyurethane, Silicone, and/or the like), any combination thereof, and/or other like materials; composites and/or other like materials; metals, such as zinc, magnesium, titanium, copper, iron, steel, carbon steel, alloy steel, tool steel, stainless steel, aluminum, any combination thereof, and/or other like materials; alloys, such as aluminum alloy, titanium alloy, magnesium alloy, copper alloy, any combination thereof, and/or other like materials; any other suitable material; and/or any combination thereof

Furthermore, the components defining any blood test strip dispenser may be purchased pre-manufactured or manufactured separately and then assembled together. However, any or all of the components may be manufactured simultaneously and integrally joined with one another. Manufacture of these components separately or simultaneously may involve extrusion, pultrusion, vacuum forming, injection molding, blow molding, resin transfer molding, casting, forging, cold rolling, milling, drilling, reaming, turning, grinding, stamping, cutting, bending, welding, soldering, hardening, riveting, punching, plating, and/or the like. If any of the components are manufactured separately, they may then be coupled with one another in any manner, such as with adhesive, a weld, a fastener (e.g. a bolt, a nut, a screw, a nail, a rivet, a pin, and/or the like), wiring, any combination thereof, and/or the like for example, depending on, among other considerations, the particular material forming the components. Other possible steps might include sand blasting, polishing, powder coating, zinc plating, anodizing, hard anodizing, and/or painting the components for example.

The embodiments and examples set forth herein were presented in order to best explain the present invention and its practical application and to thereby enable those of ordinary skill in the art to make and use the invention. However, those of ordinary skill in the art will recognize that the foregoing description and examples have been presented for the purposes of illustration and example only. The description as set forth is not intended to be exhaustive or to limit the invention to the precise form disclosed. Many modifications and variations are possible in light of the teachings above without departing from the spirit and scope of the forthcoming claims.

1. A blood test strip dispenser comprising:
a body having an inner volume and an aperture extending from the inner volume through an outer surface of the body;
a plurality of blood test strips removably retained within the inner volume of the body;
an actuating device for dispensing a single blood test strip of the plurality of blood test strips within the inner volume through the aperture, wherein the single blood test strip is a top blood test strip of the plurality of blood test strips; and
a lifting device for lifting the remaining blood test strips of the plurality of blood test strips after the single blood test strip has been dispensed, wherein lifting the remaining blood test strips comprises aligning a top blood test strip of the remaining blood test strips with the aperture.

2. The blood test strip dispenser of claim 1, wherein the actuating device comprises a control slider and a strip engagement member.

3. The blood test strip dispenser of claim 2, wherein the strip engagement member engages the single blood test strip of the plurality of blood test strips within the inner volume through the aperture and extends the single blood test strip through the aperture in response to moving the control slider from the first side toward the second side.

4. The blood test strip dispenser of claim 3, wherein the actuating device further comprises a protrusion extending from the strip engagement member, wherein the protrusion engages the single blood test strip.

5. The blood test strip dispenser of claim 4, wherein the actuating device further comprises a biasing member for biasing the actuating device toward the first side of the body.

6. The blood test strip dispenser of claim 1, wherein the lifting device comprises a support member and a lift biasing member.

7. The blood test strip dispenser of claim 6, wherein the support member supports the plurality of test strips within the inner volume of the body of the dispenser.

8. The blood test strip dispenser of claim 7, wherein the lift biasing member operates to bias the support member away from the bottom side of the body and toward the top side of the body.

9. A blood test kit comprising:
a blood test strip dispenser; and
a plurality of blood test strips, wherein the plurality of blood test strips are pre-loaded within the blood test strip dispenser or are provided in the kit for loading by a user, and wherein the blood test strip dispenser comprises:
a body having an inner volume and an aperture extending from the inner volume through an outer surface of the body, the plurality of blood test strips removably retained within the inner volume of the body;
an actuating device for dispensing a single blood test strip of the plurality of blood test strips within the inner volume through the aperture, wherein the single blood test strip is a top blood test strip of the plurality of blood test strips; and
a lifting device for lifting the remaining blood test strips of the plurality of blood test strips after the single blood test strip has been dispensed, wherein lifting the remaining blood test strips comprises aligning a top blood test strip of the remaining blood test strips with the aperture.

10. The kit of claim 9, wherein the actuating device comprises a control slider and a strip engagement member.

11. The kit of claim 10, wherein the strip engagement member engages the single blood test strip of the plurality of blood test strips within the inner volume through the aperture and extends the single blood test strip through the aperture in response to moving the control slider from the first side toward the second side.

12. The kit of claim 11, wherein the actuating device further comprises a protrusion extending from the strip engagement member, wherein the protrusion engages the single blood test strip.

13. The kit of claim 12, wherein the actuating device further comprises a biasing member for biasing the actuating device toward the first side of the body.

14. The kit of claim 9, wherein the lifting device comprises a support member and a lift biasing member.

15. The kit of claim 14, wherein the support member supports the plurality of test strips within the inner volume of the body of the dispenser.

16. The kit of claim 15, wherein the lift biasing member operates to bias the support member away from the bottom side of the body and toward the top side of the body.

17. The kit of claim 9, wherein the blood test strip dispenser with the plurality of blood test strips pre-loaded is a disposable blood test strip dispenser.

18. A method of using a blood test strip dispenser, the method comprising:
automatically aligning a top blood test strip of a plurality of blood test strips within the blood test strip dispenser with a dispensing aperture of the blood test strip dispenser;
operating an actuating device to extend the top blood test strip through the dispensing aperture;
removing the top blood test strip from the blood test strip dispenser;
automatically lifting the remaining blood test strips toward a top side of the blood test strip dispenser; and
automatically aligning a top blood test strip of the remaining blood test strips with the dispensing aperture.

19. The method of claim 18, wherein operating the actuating device comprises automatically engaging the top blood test strip of the plurality of blood test stripes within the blood test strip dispenser.

20. The method of claim 18, wherein removing the top blood test strip comprises inserting the extended blood test strip within a glucose meter directly from the blood test strip dispenser to reduce possible contamination of the extended blood test strip.
